(19) United States
(12) Patent Application Publication
(10) Pub. No.: US 2018/0190383 A1
(43) Pub. Date: Jul. 5, 2018

(54) SYSTEM, METHOD, AND APPARATUS FOR READMISSIONS RISK RATIO QUOTIENT GENERATION

(71) Applicant: Web Access, LLC, Calverton, NY (US)

(72) Inventors: Nathaniel T. Bradley, Tucson, AZ (US); William Talmage, Calverton, NY (US); Robert Dromerhauser, Bay Shore, NY (US); Joshua S. Paugh, Tucson, AZ (US)

(73) Assignee: Web Access, LLC, Calverton, NY (US)

(21) Appl. No.: 15/844,531

(22) Filed: Dec. 16, 2017

Related U.S. Application Data
(60) Provisional application No. 62/435,365, filed on Dec. 16, 2016.

(51) Int. Cl. G16H 50/30; G16H 10/60; G16H 40/20; G06Q 40/08; G06Q 10/10; G06Q 50/22

(52) U.S. Cl. CPC G16H 50/30; G16H 10/60; G06Q 50/22; G06Q 40/08; G06Q 10/10; G16H 40/20

(57) ABSTRACT

A system and method for readmissions monitoring. A sensor network is established for one or more facilities. Readmission risk indicators are determined for analysis utilize readmission data and sensor measurements measured by the sensor network. Readmissions risk analysis is performed utilizing the readmission data and the sensor measurements. Past and present readmissions data and sensor measurements are processed to determine a readmission risk quotient for one or more patients of the one or more facilities.

### PRIORITY

This application claims priority to U.S. Provisional Patent Application Ser. No. 62/435,365 entitled SYSTEM, METHOD, AND APPARATUS FOR READMISSIONS RISK RATIO QUOTIENT GENERATION filed on Dec. 16, 2017, the entirety of each prior application being incorporated by reference herein.

### BACKGROUND

### I. Field of the Disclosure

The illustrative embodiments relate to a healthcare tracking system. More specifically, but not exclusively, the illustrative embodiments relate to a system and method for communication information and data to prevent readmissions to a hospital or other care facility.

### II. Description of the Art

Preventable hospital readmissions are a significant yet avoidable cost in health care systems, costing an estimated $25-26 billion annually in the United States alone. The principal reasons cited for most hospital readmissions is poor discharge procedures and inadequate follow-up care, with a bulk of the responsibility as to cause of the readmissions falling on the side of the hospitals. In some cases, hospitals pay annual fines in relation to their total annual readmissions rates, which leaves the secondary cost of readmissions to be paid with tax payer money by Medicare and Medicaid or through private insurers, which may raise their rates to cover the unforeseen readmissions cost they incur.

### SUMMARY OF THE DISCLOSURE

The illustrative embodiments provide a system, method, and platform for readmissions monitoring. One embodiment provides a method for readmissions monitoring. A sensor network is established for one or more facilities. Readmission risk indicators are determined for analysis utilize readmission data and sensor measurements measured by the sensor network. Readmissions risk analysis is performed utilizing the readmission data and the sensor measurements. Past and present readmissions data and sensor measurements are processed to determine a readmission risk quotient for one or more patients of the one or more facilities. In another embodiment a specialized system including a processor and a memory. The processor executes a set of instructions stored in the memory to perform the method described above and otherwise herein.

In another embodiment a sensor network is established for one or more facilities. A determination of information measured by the sensor network for analysis is made. Sensor measurements are performed utilizing the sensor network. The sensor measurements are analyzed and the information to generate a readmissions risk ratio quotient. Another embodiment provides a processor and a memory storing a set of instructions, the instructions are executed by the processor to perform the described method.

Another embodiment provides a readmission monitoring system. The readmission monitoring system may include a sensor network positioned within a facility in communication with a number of sensors and devices for sensing information including at least information about patients and environments of the patients. The readmission monitoring system further includes a processing system in communication with the sensor network. The processing system analyzes the information to generate a readmission risk ratio quotient.

### BRIEF DESCRIPTION OF THE DRAWINGS

Illustrated embodiments are described in detail below with reference to the attached drawing figures, which are incorporated by reference herein, and where:

FIG. 1 is a pictorial representation of a readmissions monitoring system in accordance with an illustrative embodiment;

FIG. 2 is a flowchart of a process for generating a readmissions risk ratio quotient in accordance with illustrative embodiments;

FIG. 3 is a flowchart of process for utilizing a readmissions risk ratio quotient in accordance with an illustrative embodiment;

FIG. 4 is a flowchart of a process for generating a readmission risk ratio in accordance with an illustrative embodiment;

FIGS. 5-7 are a pictorial representation of data points utilized for generating a readmission risk ratio in accordance with an illustrative embodiment; and

FIG. 8 depicts a computing system in accordance with an illustrative embodiment.

### DETAILED DESCRIPTION OF THE DISCLOSURE

The illustrative embodiments provide a system, method, and apparatus for reducing risks of patients being readmitted to a hospital or care facility. In one embodiment, the system may track aptitude and adherence to best medical practices to reduce the risk of hospital readmission. The system may include any number of patient monitoring systems and data tracking systems that may be utilized to accumulate and track data for any number of medical service environments. The systems may be customized for various diagnostic, treatment, surgery, monitoring, and other environments to deliver data and information to any number of devices, locations, systems, equipment, platforms, users, organizations, and environments.

In one embodiment, the system is networked to be accessible through any number or type of networks enabled for secure communications. In one embodiment, the system may store the data to be displayed by any number of dashboards, applications, displays, or so forth. For example, the accumulated data may be aggregated and displayed for accessing and mitigating the risks of re-admitting a patient to a care facility for any number of medical procedures, treatments, services, or so forth. Some of the key factors for determining the readmission risk ratio may include a latent score, patient admission score, procedure score, post procedure score, perpetual score, and aggregated data score all applicable to versions of the readmission risk ratio.

The illustrative embodiments may be utilized by insurers and hospitals that are insured to provide additional layers of manageable data reconciliation and real-time assessment of risks. The illustrative embodiments provide data collection and actionable management intelligence to inform telehealth deployment decisions, maximize risk mitigation, and enhance best practices of medical professionals, groups, and facilities.

In one embodiment, a system, device, or method may generate a readmissions risk ratio quotient which is created through a weighting of various indicators across a number of medical data points. The readmission risk quotient may also be referred to as a score, percentage, relative risk, or so forth. The data points, included, but are not limited to, acute or post-acute hospital care data or any data from a procedure performed in a hospital or medical care environment. The illustrative embodiments reduce the likelihood or readmissions of a patient into a hospital or other care facility. There are any number of causes that generally result in patients being readmitted to a hospital, in addition to the standard root causes, there are countless other factors, conditions, parameters, information, and data to consider in whether a patient may require readmissions into the hospital. Although readmissions are often seen as a lapse or failure by the hospital, there are many factors that are beyond a hospitals control, including, but not limited to, varied populace, age and health demographics, socioeconomic breakup, environment, pre- and post-treatment care, and any number of other factors. There are also distinctions in readmissions that are specific to an individual's health care plan and provider.

With such a wide range of factors that contribute to the potential for readmission, unfortunately, many of the negative aspects associated with the costs and fines related to a patient's readmissions fall on hospitals, which pass the cost on to insurers and ultimately to tax payers and consumers. The illustrative embodiments utilize a new and unique system and method of performing readmissions monitoring, validation, protection, and cost reduction for hospitals and insurers through the creation of a “Readmissions Risk Ratio Quotient” that may be related to a product, service, or offering called “Readmissions Insurance.”

In one embodiment, the readmission risk ratio may compare the likelihood of a readmission for a patient (e.g., based on a visit, condition, procedure, etc.). The readmission risk ratio may be measured against the risk of readmission based on established readmission totals for various population groups or other cohorts. For example, the readmission risk for Group A may be divided by the known risk for all Groups (i.e., Group B). More simply Relative Risk (RR)=Risk in One Group (Group A)/Risk in All Other Groups. The relative risk or risk ratio compares the likelihood of dying for a certain population group against the risk of death for all other population groups. The relative risk may indicate unfair racial or social economic deaths, treatments, readmissions or so forth. The relative risk may be utilized with the readmission risk ratio quotient to determine groups at risk. Determining the relative readmission risk provides hospitals and other care facilities the opportunity to evaluate risks for a single patient compared to various known totals of past readmission data. The readmission data utilized as a reference point may be accessed from any number of government, public, private, or other services, databases, websites, collaborations, cloud systems/networks, or sharing systems. For example, a relative risk that is greater than for all groups (i.e., Group B) may indicate an increased readmission risk for the patient in Group A. The relative risk may be applied to multiple trackable healthcare data points and information (e.g., patient specific, admission, procedure, hospital/facility, recovery, sensors/medical equipment, etc.). The readmission risk quotient may also be referred to as R3Q.

The description of the embodiments as included herein is applicable across all of the systems, methods, apparatuses, devices, components, Figures, features, functions, and processes regardless of divisions or restrictions whether artificially or naturally imposed.

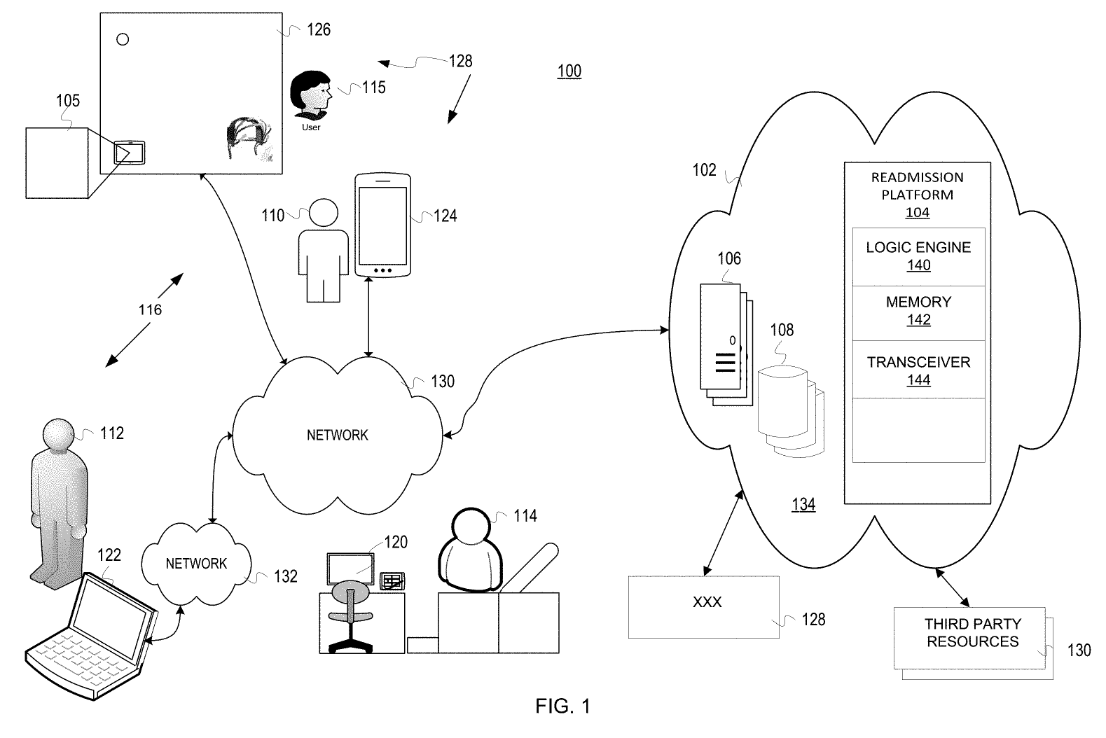

FIG. 1 is a pictorial representation of a readmissions monitoring system 100 in accordance with an illustrative embodiment. The readmissions monitoring system 100 may be installed, integrated, or utilized in any number of hospitals, locations, facilities, buildings, or areas. For example, the readmissions monitoring system 100 may represent all or portions of existing systems, equipment, and/or networks utilized in a care facility.

In one embodiment, the readmissions monitoring system 100 may include a processing system 102. Although, not specifically shown, the processing system 102 may include any number of servers, networks, databases, communications lines, routers, repeaters, cards, interfaces, intelligent devices, hubs, antennas, or so forth, such as the readmissions platform 104, servers 106, and databases 108. In one embodiment, the readmissions monitoring system 102 may represent a single or multiple locations and users 110, 112, 114, 115 (altogether users 116). The readmissions monitoring system 100 may communicate with any number of devices, such as a computer 120, a laptop 122, a wireless device 124, and sensors 126 (altogether sources 128). The sources 128 may include any number of stand-alone device, sensors, feedback machines, systems, cloud services/systems, users, parties, or so forth that may automatically or manually provide feedback to the readmissions monitoring system 100. The readmissions monitoring system 100 may also communicate with any number of medical devices, equipment, systems, databases, software, or so forth. The readmissions monitoring system 100 may utilized any number of networks, such as networks 130, 132, 134. Based on the type of locations or facilities in which the readmissions monitoring system 100 is utilized, tens, hundreds, or thousands of users may be monitored for readmissions risks.

In one embodiment, the readmissions monitoring system 100 may represent a cloud-based diagnostic system that compiles information and data from any number of sources 128. The readmissions platform 104 may generate a risk ratio quotient that determines each of the users 116 likelihood of readmission. In one embodiment, the risk ratio quotient may be tied to a product called hospital readmissions insurance. Hospital readmissions insurance is a product sold to hospitals and other caregivers as a financial protection and risk assessment tool for any demographic of patients who have a high likelihood of readmissions. As a result, hospitals, insurers, and other care facilities or groups may have the ability to identify not only the common causes of post procedure complications that potentially require readmissions, but also proves a system and method for decreasing the potential for readmissions. As a result, administrators, doctors, nurses, medical professionals, caregivers, and others may reduce the risks of readmissions based on additional criteria, processes, methods, steps, tracking systems, verifications, and so forth.

The sensors 126 may include any number of devices, components, systems, equipment, wearables, hearables, surgically implanted devices, or sensors, such as smart watches/wrist bands, monitoring equipment, brainwave interfaces, helmets, pacemakers, mobile Holter monitors, cardiac event monitors, remote cardiac service monitors, heart monitors, wireless vital sign monitors, respiratory monitors, location-aware telemonitoring sensors, blood glucose meters, asthma management, behavioral health monitors, biometric sensors, blood pressure monitors, verbal/phonetic health analysis monitors, IOT and home sensors, fall monitoring sensors, bathroom/room sensors, food safety monitors, caretaker human resource management, temperature monitors, weight monitors, sleep monitors, exercise/activity monitors, pedometers/steps and basic movement monitors, or so forth.

The sensors 126 may be worn, implanted, mounted, proximate the users 116, externally positioned, or so forth. In one embodiment, the measurements from the sources 128 may be sensors processed by each individual source 128. In another embodiment, the measurements from these sources 128 may be processed by the readmissions monitoring system 102. For example, the measurements from the wireless device 124 may be communicated through the network 130 to the readmissions monitoring system 102.

Any number of public, private, secured, wired, wireless, cloud, service, or communications based networks, such as the networks 130, 132 and a network associated with the readmissions monitoring system 102 may be utilized. In one embodiment, an application, operating system, software module, scripts, or sets of instructions may be executed by the sources 128 or readmissions platform 104 to facilitate communications, analysis, and processing of the sensor measurements to generate the readmissions risk ratio quotient.

In one embodiment, the readmissions risk ratio quotient may be generated, analyzed, compared, or otherwise processed utilizing any number of medical records, pre- or post-operative/procedure data, medical data of multiple individuals, medical claims data, home disease management monitoring data personal emergency response systems data, patient response surveys and system data, pharmacy management data, unplanned readmissions statistics, death statistics, Medicare, Medicaid, and private insurer readmissions data, healthcare provider data, healthcare insurance data, caregiver data, user/patient data, surveys, and information, and other relevant information or data however obtained. In one embodiment, the described data may be saved in one or more records of the databases 108 utilized by the readmissions platform 104. For example, the data may be associated with individual patients based on permitted access (e.g., laws, HIPAA, industry standards, best practices, etc.). In other embodiments, the data may be separated from identifying information to comply with legal, privacy, and healthcare standards and laws.

In one embodiment, the readmissions platform 104 may include a logic engine 140, a memory 142, and a transceiver 144. The logic engine 140 may be utilized to process available data and information in real time, as updated, or periodically as specified by user preferences. In one embodiment, the logic engine 140 is the logic that controls the operation and functionality of the readmissions platform 104. The logic engine 140 may include circuitry, chips, and other digital logic. The logic engine 140 or the memory 142 may also include programs, scripts, and instructions that may be implemented to operate the logic engine 140. The logic engine 140 may represent hardware, software, firmware, or any combination thereof. In one embodiment, the logic engine 140 may include one or more processors. The logic engine 140 may also represent an application specific integrated circuit (ASIC) or field programmable gate array (FPGA). In one embodiment, the logic engine 140 may execute instructions to manage the readmissions platform 104 including interactions with the components of the readmissions platform 104.

The logic engine 140 may control how and when the readmissions platform 104 generates, broadcasts, updates, and communicates the readmissions ratio quotient. The logic engine 140 may utilize any number of factors, settings, or user preferences to communicate readmissions risk quotient. In other embodiments, the logic engine 140 may readjust premiums, alerts, notifications, or other information utilized for readmissions insurance.

For example, the logic engine 140 may include a processor. The processor is circuitry or digital logic configured to execute computer-readable instructions stored on the memory 142. The memory 142 may also be internal to the processor, or provided as a separate component. The processor may be a microprocessor having a low power consumption profile, but any suitable processor may be used (e.g., digital signal processors, central processing units, ASIC, multiple core, mobile processor, wearable processor, computer processor, etc.). The processor may be a single chip or may be integrated with other computing or communications components. For example, the memory 142 may be internal to the processor or external thereto. The particular details of the processor and memory 142 need not be discussed in detail herein.

The memory 142 is a hardware element, device, or recording media configured to store data or instructions for subsequent retrieval or access. For example, the memory 142 may store measurements from the sources 128, electronic medical records, readmissions ratio quotients (and the associated data and information), and so forth. The memory 142 may represent static or dynamic memory. The memory 142 may include a hard disk, random access memory, cache, removable media drive, mass storage, or configuration suitable as storage for data, instructions, and information. In one embodiment, the memory 142 and the logic engine 140 may be integrated. The memory may use any type of volatile or non-volatile storage techniques and mediums. The memory 142 may store information related to the status of a user, readmissions platform 104, computing device, or other peripherals, such as a wireless device, smart glasses, a smart watch, a wearable device, and so forth. In one embodiment, the memory 142 may display instructions, programs, drivers, or an operating system for controlling a user interface (not shown) including one or more peripherals, touch interfaces, displays, LEDs or other light emitting components, speakers, tactile generators (e.g., vibrator), and so forth. The memory 142 may also store thresholds, conditions, user preferences, parameters, conditions, signal or processing activity, proximity data, and so forth. In one embodiment, readmissions ratio quotient may be updated in response to one or more data points exceeding a threshold or falling within a range. In other embodiments, alerts may be sent in response to the data points exceeding a threshold.

The transceiver 144 is a component comprising both a transmitter and receiver which may be combined and share common circuitry on a single housing. In one embodiment, the transceiver 144 may communicate with other devices, such as the servers 106 utilizing a wired connection (e.g., Ethernet, USB, fiber optic connectors, etc.). In other embodiments, the transceiver 144 may also communicate utilizing Bluetooth, Wi-Fi, wireless USB, ultra-wideband communications, cellular (e.g., 3G, 4G, 5G, PCS, GSM, etc.), infrared, or other suitable radio frequency standards, networks, protocols, or communications. The transceiver 144 may also be a hybrid or multi-mode transceiver that supports a number of different communications.

The components of the readmissions platform 104 may be electrically connected utilizing any number of wires, contact points, leads, busses, wireless interfaces, or so forth. In addition, the readmissions platform 104 may include any number of computing and communications components, devices or elements which may include busses, motherboards, printed circuit boards, circuits, chips, sensors, ports, interfaces, cards, converters, adapters, connections, transceivers, displays, antennas, and other similar components. Although not shown, the readmissions platform 104 may include a physical interface for connecting and communicating with other electrical components, devices, or systems. The physical interface may include any number of pins, arms, or connectors for electrically interfacing with the contacts or other interface components of external devices or other charging or synchronization devices.

Although not shown, the readmissions platform 104 itself may include any number of sensors (e.g., temperature, sound level, orientation, acceleration, motion, etc.), navigation devices (e.g., global positioning systems, wireless triangulation, etc.), or other sensors for utilization as part of the readmissions monitoring system 100.

The readmissions monitoring system 102 may further communication with third party resources 130. The third-party resources 130 may represent any number of systems, equipment, processing equipment, databases, services, or so forth. For example, the readmissions monitoring system 102 may communicate with a number of doctors, healthcare providers, insurers, hospitals, and other individuals and organizations through the third-party resources 130.

The readmissions monitoring system 102 may utilizing any number of steps or procedures to generate the readmissions ratio quotient. The readmissions ratio quotient may visually, textually, or numerically indicate various information to the user. Procedure based readmissions data points used to generate the readmissions ratio quotient may include general readmissions risk, complexity and medical errors, general and known risk of readmissions within 30 days of a hospital stay, readmissions risks associated with an unplanned extended stay in a hospital, common complications that are associated with acute events, treatments, or operations, hospital readmissions data overview from the hospital or facility performing a procedure or providing treatment, total pre-existing readmissions data from the hospital, previous readmissions data of all patients who have received the same procedure or treatment across all hospitals/facilities.

Hospital based readmissions data points used to generate the readmissions ratio quotient may include hospital excess readmissions ratios (or other applicable readmissions data), hospital readmissions penalty data, hospital discharge data, post-discharge appointments and phone follow-ups, post care wellness monitoring data, doctor patient pharmaceutical evaluation, medication compliance and monitoring, conflicts in prescribed non-pharmaceutical remedies, risk associated to logistics and ambulatory care, risks associated with health care delivery, coverage type (e.g., Medicare, Medicaid, private insurance, etc.), Internet of Things (IoT) device reports, diagnostic equipment data, brick and mortar hospital assessment, risk associated to human resources and management, visitation management and security, and so forth.

Hospital based readmissions data points used to generate the readmissions ratio quotient may include patient specific demographics versus demographics overview across the specific procedure, readmissions risk for the patient based on the procedure (e.g., high, low, numeric rating, etc.), HIPAA-Compliant patient medical database information, patient specific information (e.g., age, race, ethnicity, exercise levels, DNA results, family history, education, income, affluence, homeless or otherwise transient, etc.), environmental or socioeconomic factors, payer type of coverage (i.e. Medicare, Medicaid, Private Insurance, etc.), stay period in a hospital/facility, previously filed claims, annual primary health care or emergency room visits, patient history of drug use/abuse (e.g., alcohol, tobacco, marijuana, etc.), patient teach back interventions, and so forth. The HIPAA compliant and medical records based data utilized to generate the readmission risk quotient and baseline may include medical records data, pre-procedure and post procedure data, medical data composed of multiple individuals, medical claims data, home disease management monitoring data, personal emergency response system (PERS) data, patient response surveys and system data, pharmacy management data, Medicare, Medicaid, and Private Insurer readmission data, and medical database information. As noted, the applicable data from the various sources may be de-identified to comply with applicable laws, standards, industry practices, privacy policies, and so forth. The illustrative embodiments utilize information, data, strategies, and formulas from private, business, academic/college, State, or National Government databases including, but not limited to, healthdata.gov, cms.gov, medicare.gov, Sate Inpatient Databases (SID), Nationwide Readmissions Database (NRD), Healthcare Cost and Utilization Project (HCUP),

Post procedure based readmissions data points used to generate the readmissions ratio quotient may include home based devices, monitors, and monitoring applications, post-acute care consultation and post-secondary acute care, complications associated with procedures and treatments, and so forth.

The readmissions monitoring system 102 may be configured to generate readmissions insurance quotes and premiums based on the calculated readmissions risk ratio quotient. The readmissions monitoring system 102 may provide insurers the ability to differentiate between hospitals/facilities with a low risk for readmissions against those with a higher potential risk for unplanned secondary complications from procedures, treatments, or other resulting complications.

In one embodiment, the readmissions monitoring system 102 may be configured to automatically, semi-autonomously, or based on user input generate readmissions insurance quotes, issue verified policies, cancel policies, update quotes and policies, and collect premiums and payments.

In one embodiment, the databases 108 may include HIPAA compliant databases utilized to create probability estimates and predictive modeling. In one example, the databases 108 may include medical records absent of patient specific data, de-identified pre or post procedure data (data and information scrubbed of personal or private information), de-identified medical data composed of multiple individuals, home disease management monitoring data, personal emergency response systems (PERS) data, patient response surveys and system data, pharmacy management data, and other similar data and information. As noted the databases 108 may be updated by any number of devices, such as the servers 106, wireless device 124, laptop 122, and computer 120 through any number of different network, connections, or signals.

In one example, the computer and the user 110 may be located in a hospital. The hospital data points may include, but is not limited to: hospital and home based medical diagnostic equipment data (e.g., age of equipment, equipment software version checks, equipment error reports), post care wellness monitoring (e.g., follow up consultation with primary care physician, patient discharge, staff discharge training, training on prescribed regimen, patient signoff on discharge training, etc.), pharmaceutical evaluation of patients potential dangerous drug combination or drug interactions (e.g., current documentation of pharmaceutical regimen, pharmaceutical practices at discharge), medication compliance (e.g., medication monitoring, criteria for inclusion or exclusion in home monitoring, discharge instruction included in medication regimen, etc.), conflicts in prescribed remedies (e.g., prescribed treatment plan, at discharge, handoff from hospital to physical therapy, primary care, specialist, outpatient, in-home care, hospice, etc.), in-home post procedure monitoring (e.g., inclusion or exclusion criteria is weighted based on a hospitals utilization of home telehealth logistical data, etc.), and Internet of Things (TOT) device reports include telehealth monitoring, reporting, and communications.

The data points may also include post-secondary acute care and post-acute care consultation including health questionnaires, pharmaceutical management, daily activity monitoring, safety and security video, drug rehabilitation, and video diagnostic consultation.

Additionally, the data points may potentially exclude identified patient information geographic subdivisions smaller than a state (e.g., street address, city and ZIP code, etc.), all dates that are related to an individual (e.g., date of birth, admission, etc.), telephone numbers, fax numbers, email addresses, Social Security numbers, medical record numbers, allergies, health plan beneficiary numbers, account numbers, certificate/license numbers, vehicle identifiers and serial numbers (e.g., license plate numbers), device identifiers and serial numbers, Web universal locators (URLs), IP address numbers, biometric identifiers (e.g., fingerprints and voice prints), full-face photographic images, other unique identifying numbers (e.g., characteristics or codes), or so forth.

The illustrative embodiments provide a system, method, network, and platform that may be utilized to reduce costs due to readmissions which are extensive (e.g., $26 billion with $17 billion in costs for Medicare alone). Readmissions insurance may provide a cost-effective way of assessing, managing, and averting risk due to readmission. The illustrative embodiments solve the problem of assessing which procedures, health diagnostic tools, and post procedure practices reduce hospital costs. The readmission rate information may be particularly useful for conditions, such as heart failure, acute myocardial infarction, pneumonia, cardiovascular disease, heart disease, respiratory disease, cancer, pediatric ailments, and so forth.

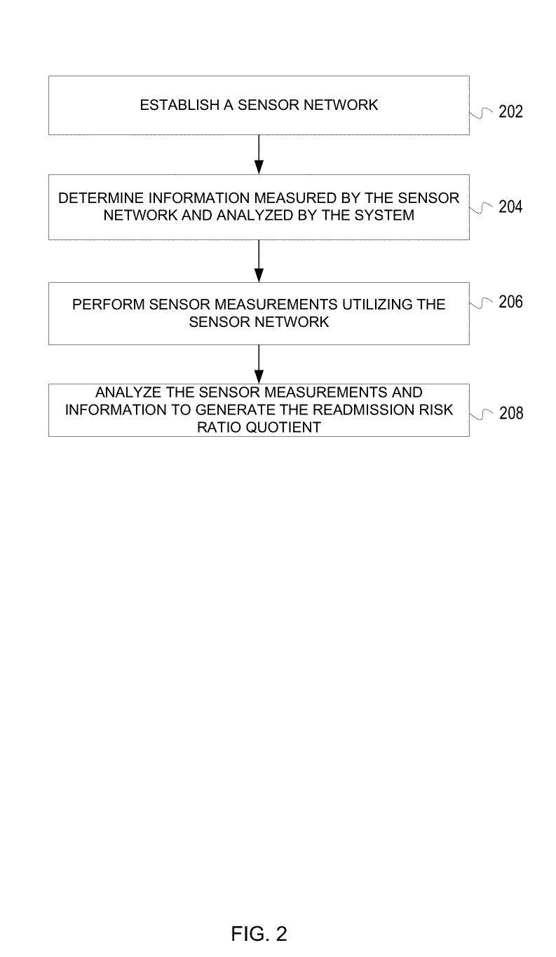

FIG. 2 is a flowchart of a process for generating a readmissions risk ratio quotient in accordance with illustrative embodiments. The process of FIG. 2 may be performed by one or more devices, systems, equipment, or components, such as the readmissions monitoring system 102 of FIG. 1. In one embodiment, the readmissions monitoring system may represent a cloud based system. The readmission monitoring system may communicate with numerous other cloud or network based systems, devices, equipment, users, and so forth.

In one embodiment, the process may begin by establishing a sensor network (step 202). The sensor network may include any number of wearable, implantable, environment, or other devices, components, or sensors that measure information relevant to a user or an environment of the user, such as activity, actions, voice frequency and amplitude, body position, location, temperature, blood pressure, heart rate, external temperature, and so forth as enumerated herein. The sensor network may be established across multiple locations, environments, and/or facilities that one or more users may visit during a given day. The sensor network may be established by an individual, group, or service provider. For example, the sensor network may be established for a number of patients at a hospital that may encompass numerous buildings, wings, divisions, departments, or so forth. The sensor network may communicate with any number of databases, services, networks, systems, or repositories that are pre-populated with applicable local, regional, national, and worldwide readmissions data, statistics, and reports. The readmission risk data may be for a day, week, month, year, custom time period, or so forth. In one embodiment, one or more sensors may be assigned to each new patient admitted. The sensor may be embedded in a patient's medical bracelet, clothing (e.g., shirt, headband, etc.), footwear, earwear, medical records, assigned equipment (e.g., bed, heart monitor, etc.), or other object.

Next, the system determines information measured by the sensor network and analyzed by the system (step 204). The information may include any number of data points and information retrieved or measured from the user, medical or care professionals, environment, or otherwise determined. The information may also be retrieved from any number of sources including servers, databases, third-parties, services, or so forth. The information may be specified by user preferences, parameters, configurations, or so forth.

Next, the system performs sensor measurements utilizing the sensor network (step 206). The sensors measurements may be performed at any time for real-time or subsequent analysis. In one embodiment, the patient's data is tracked by doctors, nurses, medical professionals, data entry clerks, family, or others based on data accumulated from the sensors or network devices. In one embodiment, the patient's data is de-identified to comply with applicable laws and to ensure privacy protection.

Next, the system analyzes the sensor measurements and information to generate a readmissions risk ratio quotient (step 208). The readmissions risk ratio quotient may be generated for the first time or updated based on new information including sensor measurements. For example, all or portions of the process of FIG. 2 may be repeated to update the readmissions risk ratio quotient (e.g., steps 206-208 unless there is a change to the sensor network or the information requiring steps 202 and/or 204 to be performed again).

In one embodiment, each factor that affects readmissions may be utilized to generate a weighted factor/average that is evaluated. For example, an overall readmission risk ratio may be generated from a latent score, patient admission score, procedure score, post procedure score, perpetual score, and aggregated data score all applicable to the same or different versions or iterations of the readmission risk ratio. In one embodiment, the readmissions risk ratio quotient may be a percentage showing likelihood that a user/patient will be readmitted to a hospital or facility.

The patient readmission risk ratio or score may be tracked in the form of each user's de-identified patient diagnosis (e.g., certified coding category number or name) and any subsequent diagnosis that the patient may receive. The de-identified patient admission data may be weighted and scored against all known facility, local, regional, and national readmissions statistics. The admission and readmission ratio quotient scores may be tracked and tallied at various levels (e.g., locally, facility, group of facilities, network, county, state, country, region, worldwide, etc.).

The readmission risk ratio quotient may be utilized by a single hospital or multiple facilities. The readmission risk ratios may have been previously aggregated or may be aggregated at any time. In one embodiment, benchmarks may be created based on the hospital's previous annual readmission statistics and any past readmission data, or other indicators related to the hospital and their readmission totals. In another example, the readmissions risk ratio quotient may be received from a separate system for utilization. In one embodiment, each participating hospital's past annual readmission risk ratio totals may be weighted and scored against all known local, regional, and national readmissions statistics to generate the readmission risk ratio. The calculation may provide a latent readmission risk ratio score that reflects the hospital's past annual readmission data when compared to past and current annual readmission risk averages.

In one embodiment, the sensor data for each patient and the hospital's current aggregated total readmission risk scores for each patient admitted are accumulated at a network level. The aggregated total readmission risk scores may be assigned a perpetual readmission risk score. The perpetual readmission risk score may be an accumulation of data (e.g., de-identified patient data) based on the daily, weekly, monthly, and yearly number of all patients seen at a hospital. The perpetual readmission risk score may be measured in set intervals across the entire patient demographic from the patient data that indicates zero or low readmission risk to patient data that indicates a generally higher risk of readmissions.

The sensors may aggregate, track, and measure readmission risk quotients, scores, or indicators for each new admission to a facility, such as a hospital. The measured risk indicators may include, but are not limited to, admission cause, procedure risk, hospital data, patient data, and post procedure data. Based on the aggregated data from each of the risk indicator categories, the sensor data may be utilized by a network system to identify individuals, populations, cohorts, or groups that have an elevated potential for readmissions. The combination of risk indicators is factored into the aggregated RRQ score which may be utilized for any number of purposes, including process improvement, risk analysis, insurance, and so forth.

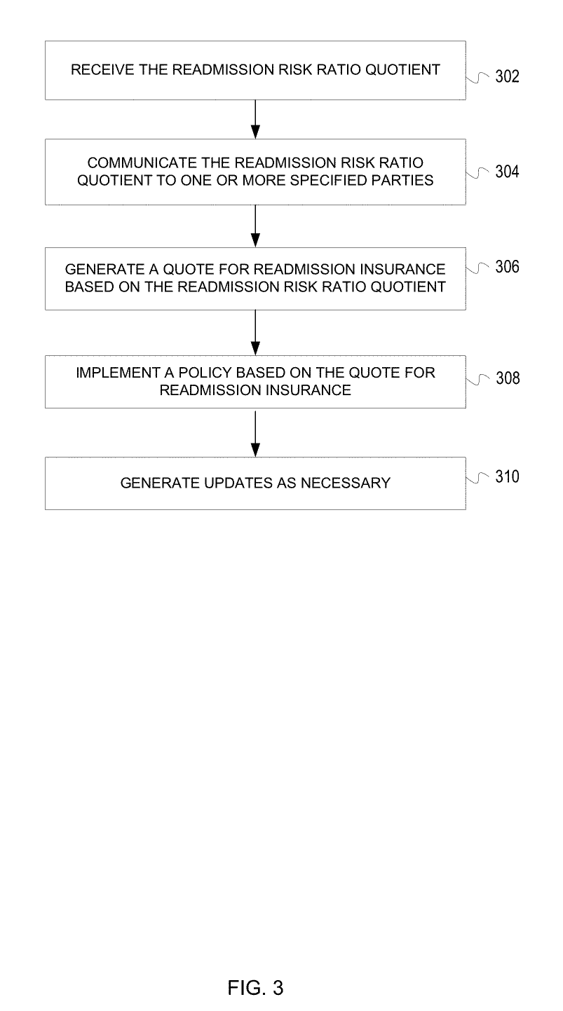

Turning now to FIG. 3, illustrating a flowchart of a process for utilizing a readmissions risk ratio quotient in accordance with an illustrative embodiment. In one embodiment, the process of FIG. 3 may be performed after or in conjunction with the process of FIG. 2. The process of FIG. 3 may be performed by a system or device utilized in FIG. 2 or by a separate or third-party device or system. In one embodiment, the process may begin by receiving the readmissions risk ratio quotient (step 302). The readmissions risk ratio quotient may be received in response to being generated or retrieved from one or more available sources. For example, the readmissions risk ratio quotient may be received from logic of the system. An available pool of past and current readmission risk ratios may be retrieved for the local area, region, nation, or world.

Next, the system communicates the readmissions risk ratio quotient to one or more specified parties. The specified parties may include users, administrators, systems, devices, third parties, or so forth. The readmissions risk ratio quotient may be included in any number of graphics, visuals, alerts, or messages. In one embodiment, the system may communicate the readmissions risk ratio quotient in response to changes in the readmissions risk ratio quotient that exceed one or more thresholds. The readmissions risk ratio quotient may also be received and communicated between software modules.

Next, the system generates a quote for readmissions insurance based on the readmissions risk ratio quotient (step 306). In one embodiment, updates to the readmissions risk ratio quotient may be received at any time and used to update the readmissions insurance quote. The quote may include physical or electronic quote documentation. The quote may be communicated through a web interface, program or mobile app, or any number of interfaces. The latent or past readmission risk ratio score may be factored into the initial insurance premium (or applicable rates and terms) the hospital is quoted for payment based on the aggregated information. The patient admission cause and readmission risk ratio quotient scores may be factored into the insurance premium and rate that a facility may pay. The premiums, rate, and deductible may be adjusted lower or higher based on the annual or monthly totals for the readmission risk ratio quotient determined for the hospital, facility, or group.

Next, the system implements a policy based on the quote for readmissions insurance (step 308). During step 308, the system may receive user input as required (e.g., signatures, payment information, updated information, etc.), process payments, and communicate policies, certificates, and updates. In one embodiment, to implement the policy, an authorized user or administrator of the covered facility/hospital may be required to sign

Next, the system generates updates as necessary (step 310). For example, updated information including sensor measurements, user input, data, and so forth may be received. The readmissions risk ratio quotient may be updated in real-time (or near real-time) or at designated intervals, such as hourly, daily, weekly, monthly, yearly, or any specified period. The updates may include updated quotes, policies, warnings, alerts, payment information, and so forth. During step 310 additional information may be sent for review, signatures, verification, or so forth.

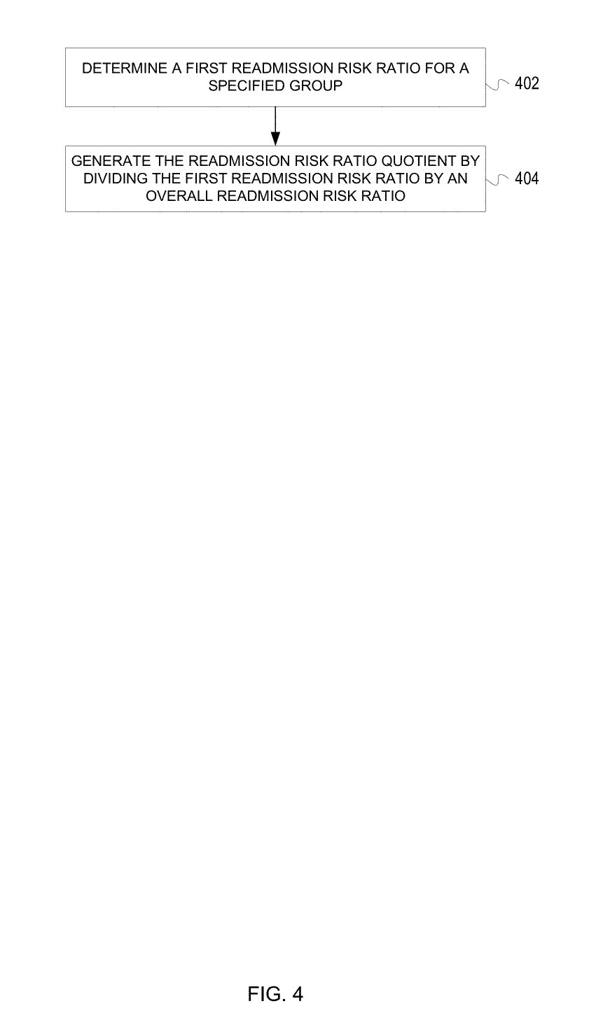

FIG. 4 is a flowchart of a process for generating a readmission risk ratio in accordance with an illustrative embodiment. The process of FIG. 4 may be performed utilizing the readmission monitoring system of FIG. 1 or other similar devices and systems. In one embodiment, the process may being by determining a first readmission risk ratio for a specified group (step 402). The readmission risk ratio may represent a readmission risk ratio quotient, score, percentage, status, indicator, value, or other information. The specified group may associated with the readmission risk ratio represent a facility, multiple facilities, regions, demographics, cohorts, groups of people (e.g., linked by disease, treatment, etc.), or so forth.

Next, the system generates the readmission risk ratio quotient by dividing the first readmission risk ration by an overall readmission risk ratio (step 404). The overall readmission risk ratio may represent an overall readmission risk ratio associated with the first readmission risk ratio (e.g., applicable aggregations of associated groups).

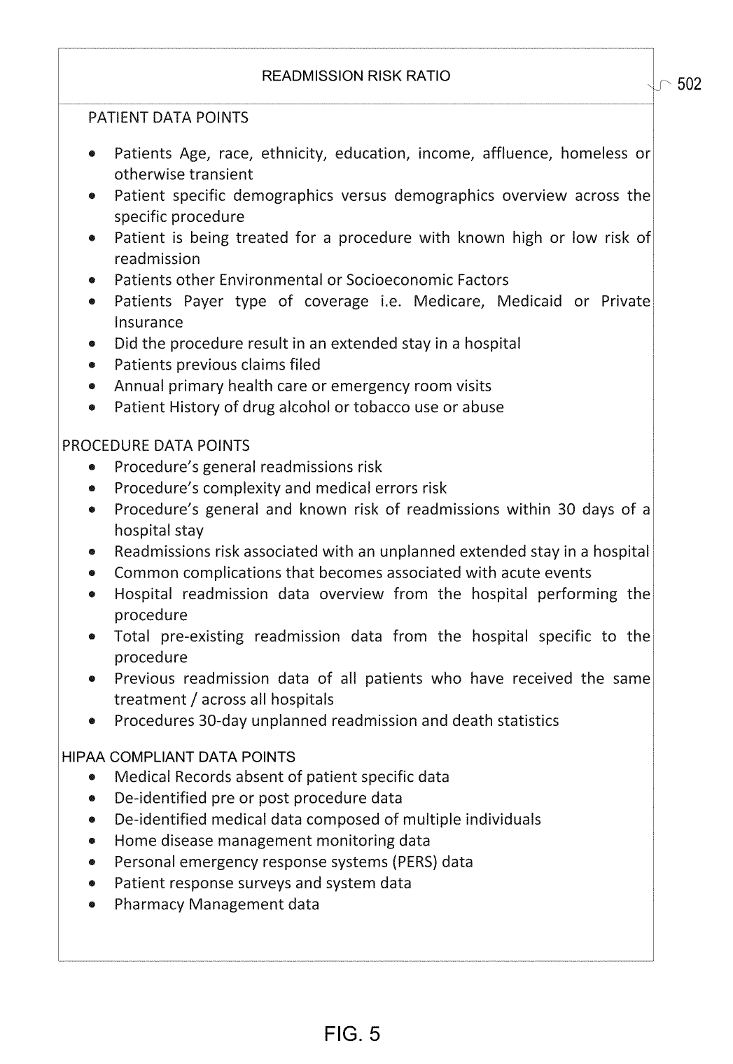

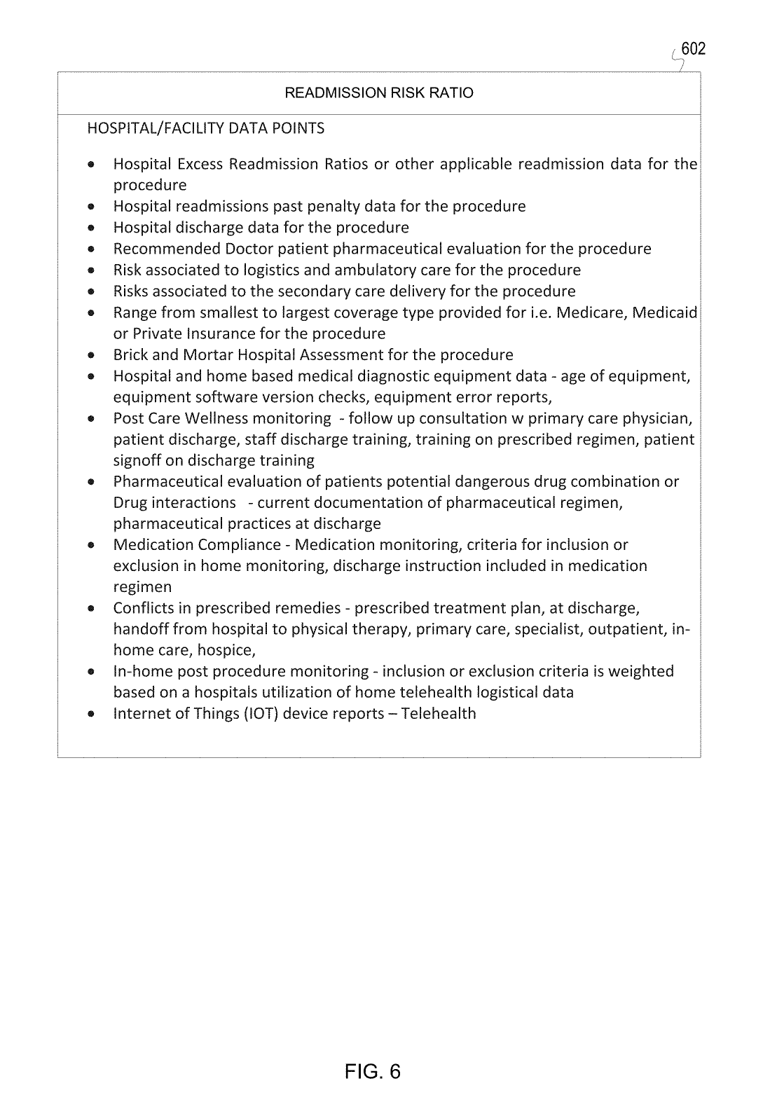

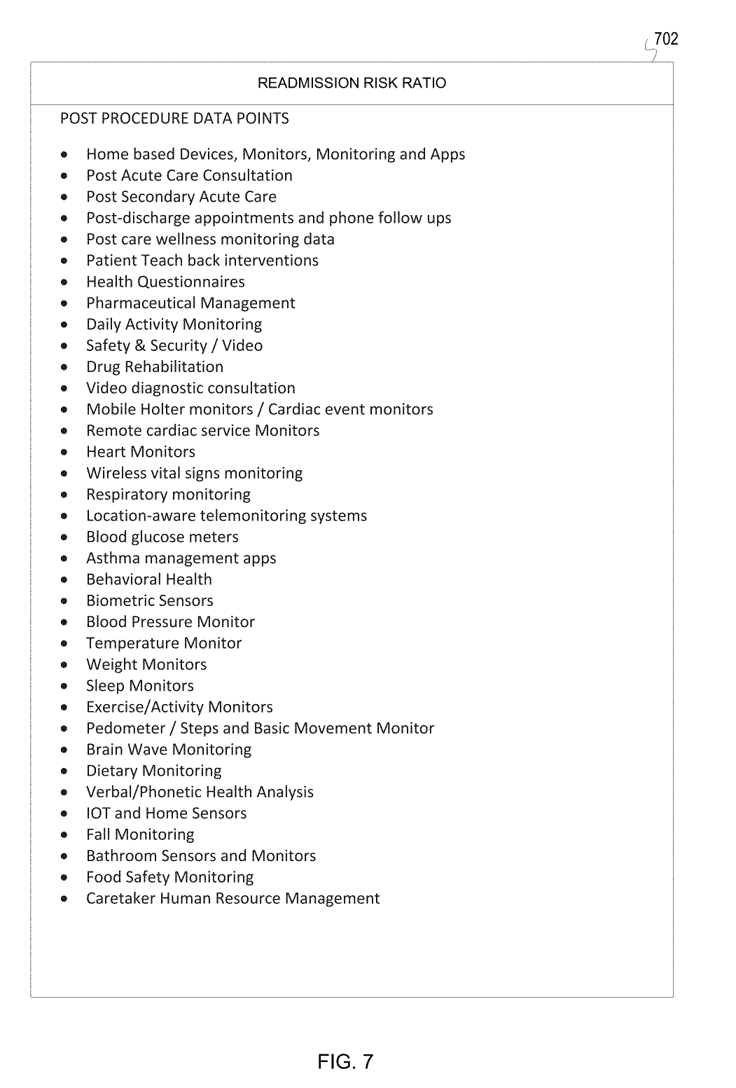

FIGS. 5-7 are a pictorial representation of data points 502, 602, 702, utilized for generating a readmission risk ratio in accordance with an illustrative embodiment. The data points 502, 602, 702 may represent patient data points, procedure data points, HIPAA compliant data points, hospital/facility data points, and post procedure data points.

The illustrative embodiments may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, embodiments of the inventive subject matter may take the form of a computer program product embodied in any tangible medium of expression having computer usable program code embodied in the medium. The described embodiments may be provided as a computer program product, or software, that may include a machine-readable medium (non-transitory storage medium) having stored thereon instructions, which may be used to program a computing system (or other electronic device(s)) to perform a process according to embodiments, whether presently described or not, since every conceivable variation is not enumerated herein. A machine-readable medium includes any mechanism for storing or transmitting information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read only memory (ROM); random access memory (RAM); erasable programmable memory (e.g., EPROM and EEPROM); flash memory; or other types of medium suitable for storing electronic instructions. In addition, embodiments may be embodied in an electrical, optical, acoustical or other form of propagated signal (e.g., carrier waves, infrared signals, digital signals, etc.), or wireline, wireless, or other communications medium.

Computer program code for carrying out operations of the embodiments may be written in any combination of one or more programming languages, including an object oriented programming language such as Java, Smalltalk, C++ or the like and conventional procedural programming languages, such as the “C” programming language or similar programming languages. The program code may execute entirely on a user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN), a personal area network (PAN), or a wide area network (WAN), or the connection may be made to an external computer (e.g., through the Internet using an Internet Service Provider).

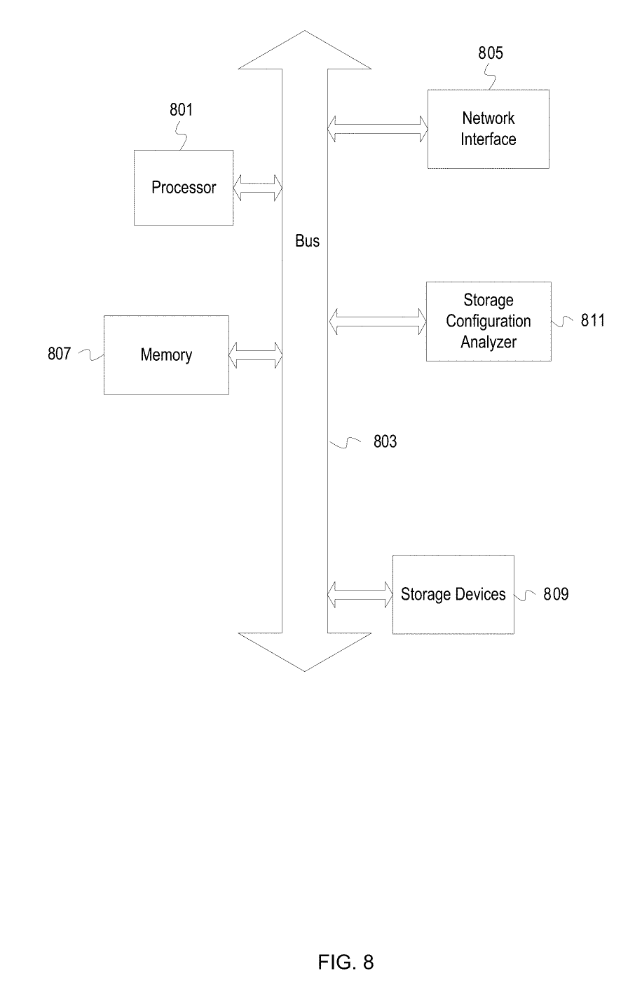

FIG. 8 depicts a computing system 800 in accordance with an illustrative embodiment. For example, the computing system 800 may represent a system or device, such as the processing system 101 of FIG. 1. The computing system 800 includes a processor unit 801 (possibly including multiple processors, multiple cores, multiple nodes, and/or implementing multi-threading, etc.). The computing system includes memory 807. The memory 807 may be system memory (e.g., one or more of cache, SRAM, DRAM, zero capacitor RAM, Twin Transistor RAM, eDRAM, EDO RAM, DDR RAM, EEPROM, NRAM, RRAM, SONOS, PRAM, etc.) or any one or more of the above already described possible realizations of machine-readable media. The computing system also includes a bus 803 (e.g., PCI, ISA, PCI-Express, HyperTransport®, InfiniBand®, NuBus, etc.), a network interface 806 (e.g., an ATM interface, an Ethernet interface, a Frame Relay interface, SONET interface, wireless interface, etc.), and a storage device(s) 809 (e.g., optical storage, magnetic storage, etc.). The system memory 807 embodies functionality to implement all or portions of the embodiments described above. The system memory 807 may include one or more applications or sets of instructions for implementing tracking, monitoring, associating, or generation of inaudible signals. In one embodiment, an inaudible signal engine may be utilized to communicate with one or more transmitters or sensors. The inaudible signal engine may be stored in the system memory 807 and executed by the processor unit 802. Code may be implemented in any of the other devices of the computing system 800. Any one of these functionalities may be partially (or entirely) implemented in hardware and/or on the processing unit 801. For example, the functionality may be implemented with an application specific integrated circuit, in logic implemented in the processing unit 801, in a co-processor on a peripheral device or card, etc. Further, realizations may include fewer or additional components not illustrated in FIG. 8 (e.g., video cards, audio cards, additional network interfaces, peripheral devices, etc.). The processor unit 801, the storage device(s) 809, and the network interface 805 are coupled to the bus 803. Although illustrated as being coupled to the bus 803, the memory 807 may be coupled to the processor unit 801. The computing system 800 may further include any number of optical sensors, accelerometers, magnetometers, microphones, gyroscopes, temperature sensors, and so forth for verifying user biometrics, or environmental conditions, such as motion, light, or other events that may be associated with the transmitters (e.g., chip, tag, etc.), sensors, user, or their environment.

The features, steps, and components of the illustrative embodiments may be combined in any number of ways and are not limited specifically to those described. In particular, the illustrative embodiments contemplate numerous variations in the smart devices and communications described. The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of embodiments, processes or methods of the invention. It is understood that any other modifications, substitutions, and/or additions may be made, which are within the intended spirit and scope of the disclosure. For the foregoing, it can be seen that the disclosure accomplishes at least all of the intended objectives.

The previous detailed description is of a small number of embodiments for implementing the invention and is not intended to be limiting in scope. The following claims set forth a number of the embodiments of the invention disclosed with greater particularity.

What is claimed is:

1. A readmissions monitoring method, comprising:
establishing a sensor network for one or more facilities for performing sensor measurements;
determining readmission risk indicators for analysis utilizing readmission data and the sensor measurements measured by the sensor network;
performing readmissions risk analysis using utilizing the readmission data and sensor measurements;
processing past and present readmission data and sensor measurements to determine a readmission risk quotient for one or more patients of the one or more facilities.

2. The method of claim 2, further comprising:
performing sensor measurements utilizing the sensor network.

3. The readmissions monitoring method of claim 1, further comprising:
communicating the readmissions risk ratio quotient to one or more users or devices.

4. The readmissions monitoring method of claim 1, further comprising:
generating a quote for readmissions insurance for a party associated with the one or more facilities based on the readmissions risk ratio quotient compared with a readmission risk quotient for other groups.

5. The readmissions monitoring method of claim 4, further comprising:
receiving acceptance of the quote from the party; and
issuing a readmissions insurance policy for the readmissions insurance to the party in response to the readmissions risk ratio quotient.

6. The readmissions monitoring method of claim 1, further comprising:
updating the readmissions risk ratio quotient in response to additional readmission data and sensor measurements.

7. The readmissions monitoring method of claim 6, further comprising:
updating a policy for readmissions insurance for a party in response to updating the readmissions risk ratio quotient, wherein the policy includes a price paid by the party.

8. The readmissions monitoring method of claim 1, further comprising:
sending one or more alerts based on updates or changes to the readmissions risk ratio quotient.

9. The readmissions monitoring method of claim 1, wherein the readmissions risk ratio quotient includes a plurality of data points including medical records, procedure data, readmission data, hospital or facility data, patient data, post care data, and acute care data.

10. The readmissions monitoring method of claim 8, further comprising:
indicating which of the plurality of data points most significantly affect readmissions risks.

11. A readmissions monitoring system, comprising:
a sensor network positioned within a facility in communication with a plurality of sensors and devices for sensing information including at least readmission data associated with the information about patients and environments of the patients;
a processing system in communication with the sensor network, wherein the processing system analyzes the information and readmission data to generate a readmissions risk ratio quotient.

12. The readmissions monitoring system of claim 11, wherein the facility includes one or more facilities operated by a party.

13. The readmissions monitoring system of claim 11, wherein the processing system further sends one or more communications including at least the readmissions risk ratio quotient.

14. The readmissions monitoring system of claim 11, wherein the processing system furthers generates a readmissions insurance policy for a party operating the facility utilizing at least the readmissions risk ratio quotient.

15. The readmissions monitoring system of claim 11, wherein the information includes a plurality of data points including medical records, procedure data, the readmission data, facility or location data, patient data, post care data, and acute care data.

16. The readmissions monitoring system of claim 11, wherein the processing system generates a quote for readmissions insurance for a party associated with the facility based on the readmissions risk ratio quotient, receives acceptance of the quote from the party, issues a readmissions policy for the readmissions insurance to the party in response to the readmissions risk ratio quotient.

17. The readmissions monitoring system of claim 10, wherein the processing system further updates the readmissions risk ratio quotient in response to updated information, and sends one or more alerts to one or more specified users in response to the readmissions risk ratio quotient exceeding one or more thresholds.

18. A readmissions monitoring system, comprising:
a processor for executing a set of instructions;
a memory for storing the set of instructions;
one or more sensors for measuring information associated with one or more patients in a facility;
a transceiver for receiving readmission data as well as sensor measurements from the one or more sensors, wherein the set of instructions are executed by the processor to analyze readmission data and sensor measurements information measured by the sensor network, and process the sensor measurements and the readmission data to generate a readmissions risk ratio quotient.

19. The readmissions monitoring system of claim 18, wherein the set of instructions is further executed to manage a readmissions insurance for the facility in response to the readmissions risk ratio quotient.

20. The readmissions monitoring system of claim 17, wherein the information includes a plurality of data points including medical records, procedure data, facility data, patient data, post care data, and acute care data.

21. The readmissions monitoring system of claim 17, wherein the set of instructions are further executed to:
send one or more communications including at least the readmissions risk ratio quotient.

22. The readmissions monitoring system of claim 18, wherein the set of instructions are further executed to generate a quote for readmissions insurance for a party associated with the facility based on the readmissions risk ratio quotient, receive acceptance of the quote from the party, and issue a readmissions policy for the readmissions insurance to the party in response to the readmissions risk ratio quotient.
